(19) United States
(12) Patent Application Publication
(10) Pub. No.: US 2026/0049142 A1
(43) Pub. Date: Feb. 19, 2026

(54) ANTI-HUMAN ICOS ANTIBODIES FOR USE IN IMMUNOHISTOCHEMISTRY (IHC) PROTOCOLS AND FOR DIAGNOSING CANCER

(71) Applicant: AGILENT TECHNOLOGIES, INC., Santa Clara, CA (US)

(72) Inventors: Morten Draeby SORENSEN, Santa Clara, CA (US); Tine HAGEDORN-OLSEN, Santa Clara, CA (US); Morten Alder SCHULZ, Santa Clara, CA (US)

(73) Assignee: AGILENT TECHNOLOGIES, INC., Santa Clara, CA (US)

(21) Appl. No.: 19/102,961

(22) PCT Filed: Sep. 22, 2023
(86) PCT No.: PCT/US2023/033479

§ 371 Date: Feb. 11, 2025

Related U.S. Application Data
(60) Provisional application No. 63/409,076, filed on Sep. 22, 2022.

(51) Int. Cl. C07K 16/28; G01N 33/574

(52) U.S. Cl. CPC C07K 16/2818; G01N 33/57492; G01N 2333/70521

(57) ABSTRACT

In alternative embodiments, provided are chimeric, synthetic or recombinant anti-human ICOS protein or polypeptide (also called inducible T-cell co-stimulator, or Cluster of Differentiation-278, or CD278) antibodies (Abs), including products of manufacture and kits comprising them, and methods for making and using them, including for example their use in the detection or diagnosis, and treatment, of a cancer, or other diseases or conditions involving expression of ICOS. In alternative embodiments, anti-ICOS proteins (for example, antibodies) as provided herein are used together with an agent for determining whether ICOS expression or activity is present, increased, reduced or absent. In alternative embodiments, anti-ICOS antibodies as provided herein are used in the diagnosis and/or treatment of a cancer or a tumor.

### RELATED APPLICATIONS

This PCT international utility patent application claims the benefit of priority under 35 U.S.C. § 119 (e) to U.S. Provisional Patent Application Ser. No. (USSN) 63/409,076, Sep. 22, 2022. The aforementioned application is expressly incorporated herein by reference in its entirety and for all purposes.

### TECHNICAL FIELD

This invention generally relates to immunohistochemistry (IHC) and cancer diagnosis and treatment. In alternative embodiments, provided are chimeric, synthetic or recombinant anti-human ICOS protein or polypeptide (also called inducible T-cell co-stimulator, or Cluster of Differentiation-278, or CD278) antibodies (Abs), including products of manufacture and kits comprising them, and methods for making and using them, including for example their use in the detection or diagnosis, and treatment, of a cancer, or other diseases or conditions involving expression of ICOS. In alternative embodiments, anti-ICOS proteins (for example, antibodies) as provided herein are used together with an agent for determining whether ICOS expression or activity is present, increased, reduced or absent. In alternative embodiments, anti-ICOS antibodies as provided herein are used in the diagnosis and/or treatment of a cancer or a tumor, for example, a carcinoma (optionally a squamous carcinoma), a mamma carcinoma, a colon carcinoma or a colorectal cancer, a melanoma (optionally a malignant melanoma) or a multiple myeloma, a plasmacytoma, a lymphoma, a bladder or urothelial cancer, a cervical cancer, an ovarian cancer, an esophageal cancer or an esophageal squamous; a malignant pleural mesothelioma, a prostate cancer, a microsatellite instability-high/deficient mismatch repair tumor, a human Papilloma Virus-positive or Epstein-Barr positive tumor, a hepatocellular carcinoma or liver cancer, a lung cancer (optionally a non-small cell lung cancer (NSCLC)), a gastric cancer, a renal or kidney cancer, a pancreatic cancer, a breast cancer (optionally a triple-negative breast cancer), a lymphoma or a leukemia, or a mycosis fungoides.

### BACKGROUND

ICOS protein, also called inducible T-cell co-stimulator, or Cluster of Differentiation-278, or CD278, is in the CD28 and CTLA-4 cell-surface receptor family. ICOS protein forms homodimers, playing an important role in cell-cell signaling, immune responses and regulation of cell proliferation. ICOS is a costimulatory receptor for T-cell enhancement.

ICOS+ T cells have been found in in tumor tissues, and anti-CTLA-4 treatment in mice with ICOS+ tumors resulted in tumor rejection in 80% to 90% of subjects, but in gene-targeted mice that were deficient for either ICOS or its ligand (ICOSLG), the efficacy was less than 50%.

ICOS activation might potentiate the effect of an inhibitory checkpoint blockade, while its neutralization could decrease the function of immunosuppressive Tregs and inhibit lymphoid tumor cells expressing Tfh markers.

Vopratelimab (JTX-2011) is an investigational humanized IgG1κ agonist mAb that specifically binds to ICOS and is designed to augment an antitumor immune response. The mechanism of action of vopratelimab requires the initial priming of T cells followed by upregulation of ICOS expression on the cell surface of CD4 T cells, after which vopratelimab engagement results in T cell proliferation and sustained activation.

### SUMMARY

In alternative embodiments, provided are isolated or purified antibodies (Ab), or antigen (Ag) binding fragments thereof, or monomeric or dimeric antigen binding proteins (ABP), capable of specifically binding a human ICOS (Inducible T-cell costimulatory) protein, or human CD278, polypeptide,
wherein the isolated or purified Ab, or Ag binding fragment thereof, or monomeric or dimeric ABP comprises:
(a) a heavy chain variable region (VH) comprising:
   (1) an amino acid sequence comprising the three CDR1, CDR2 and CDR3 complementarity determining regions (CDRs) of SEQ ID NO:1, or CDR1 amino acid (aa) residues GFSLSSYG (residues 25-32 of SEQ ID NO:1), CDR2 aa residues INSDHST (residues 50 to 56 of SEQ ID NO:1), and CDR3 aa residues ARSYGIGSIF (residues 93-102 of SEQ ID NO:1), or
   (2) amino acid sequences having at least about 70%, 75%, 80%, 85%, 90%, 95%, 98% sequence identity, or between about 70% to 100% sequence identity, to each of the three CDR1, CDR2 and CDR3 complementarity determining regions (CDRs) of SEQ ID NO:1, or CDR1 amino acid (aa) residues GFSLSSYG (residues 25-32 of SEQ ID NO:1), CDR2 aa residues INSDHST (residues 50 to 56 of SEQ ID NO:1), and CDR3 aa residues ARSYGIGSIF (residues 93-102 of SEQ ID NO:1), or
   (3) an amino acid sequence having at least about 70%, 75%, 80%, 85%, 90%, 95%, 98% sequence identity, or between about 70% to 100% sequence identity, to SEQ ID NO:1, or an amino acid sequence having complete sequence identity to SEQ ID NO:1; or
(b) a light chain variable region (VL) comprising:
   (1) an amino acid sequence comprising the three CDR1, CDR2 and CDR3 complementarity determining regions (CDRs) of SEQ ID NO:2, or CDR1 amino acid (aa) residues KSVYNNNQ (residues 27-34 of SEQ ID NO:2), CDR2 aa residues EAF (residues 52 to 54 of SEQ ID NO:2), and CDR3 aa residues AAVYSDDSDNS (residues 91-101 of SEQ ID NO:2), or
   (2) amino acid sequences having at least about 70%, 75%, 80%, 85%, 90%, 95%, 98% sequence identity, or between about 70% to 100% sequence identity, to each of the three CDR1, CDR2 and CDR3 complementarity determining regions (CDRs) of SEQ ID NO:2, or CDR1 amino acid (aa) residues KSVYNNNQ (residues 27-34 of SEQ ID NO:2), CDR2 aa residues EAF (residues 52 to 54 of SEQ ID NO:2), and CDR3 aa residues AAVYSDDSDNS (residues 91-101 of SEQ ID NO:2); or
   (3) an amino acid sequence having at least about 70%, 75%, 80%, 85%, 90%, 95%, 98% sequence identity, or between about 70% to 100% sequence identity, to SEQ ID NO:2, or an amino acid sequence having complete (100%) sequence identity to SEQ ID NO:2; or
(c) the heavy chain variable region (VH) of (a) and the light chain variable region (VL) of (b).

In alternative embodiments, of isolated or purified antibodies (Ab), or antigen (Ag) binding fragments thereof, or monomeric or dimeric antigen binding proteins (ABP) as provided herein:
the isolated or purified antibodies (Ab), or antigen (Ag) binding fragments thereof, or monomeric or dimeric antigen binding proteins (ABP) are fabricated as or in the form of:
an antigen-binding fragment (Fab, or an Ab fragment having just one constant and one variable domain of each of an Ab heavy and light chain),
a F(ab′)₂ (or an Ab digested by pepsin yielding two fragments: a F(ab′)₂ fragment and a pFc′ (pepsin cleavage Fc) fragment),
a Fab′ (a single chain of a F(ab′)₂ fragment),
a single-chain variable fragment (scFv) (or a fusion protein of a variable region of an Ab heavy and light chain connected together with a linker peptide optionally of about ten to about 25 amino acids in length),
a (scFv)₂, or a di-scFv or a bi-scFv, or a single peptide chain having two variable heavy and two variable light regions yielding tandem scFv,
a minibody (or a fusion protein of a variable region of an Ab heavy and light chain connected together with an alkyl group, optionally a methyl or an ethyl group)
a diabody (or an scFv with a linker peptide too short (optionally about five amino acids) for the two variable regions to fold together forcing the scFvs to dimerize), a triabody or a tetrabody (or an scFv with a linker peptide too short (optionally about one or two amino acids) for the two variable regions to fold together forcing the scFvs to trimerize or tetramize),
a single-domain antibody (dAB) (or a single variable region of an Ab heavy or Ab light chain),
a plurality of complementarity determining region (CDR) fragments, or
a multispecific antibody formed from two or more antibody fragments;
the heavy chain variable region (VH), if present, comprises:
an amino acid sequence:

(SEQ ID NO: 1)
QSLEESGGRLVTPGTPLTLTCTVSGFSLSSYGVSWVRQAPGKGLEWIGI
INSDHSTYYAKWAKGRFTISKTSTTVDLKITSPTTEDTATYFCARSYGI
GSIFWGPGTLVTVSS,

or

SEQ ID NO:1 having one or more amino acid substitutions, additions (insertions) or deletions, and the recombinant Ab, or Ag binding fragment thereof, or monomeric or dimeric ABP retains its ability to specifically bind to the human ICOS protein or polypeptide;
the one or more amino acid substitutions in the heavy chain variable region (VH) comprise one or more conservative amino acid substitutions, and optionally the one or more conservative amino acid substitutions comprise: replacement of an aliphatic amino acid with another aliphatic amino acid; replacement of a serine with a threonine or vice versa; replacement of an acidic residue with another acidic residue; replacement of a residue bearing an amide group with another residue bearing an amide group; exchange of a basic residue with another basic residue; or replacement of an aromatic residue with another aromatic residue;
the heavy chain variable region further comprises at least a portion of a heavy chain constant region, and optionally the heavy chain constant region comprises an amino acid sequence:

(SEQ ID NO: 3)
GQPKAPSVFPLAPCCGDTPSSTVTLGCLVKGYLPEPVTVTWNSGTLTNG
VRTFPSVRQSSGLYSLSSVVSVTSSSQPVTCNVAHPATNTKVDKTVAPS
TCSKPTCPPPELLGGPSVFIFPPKPKDTLMISRTPEVTCVVVDVSQDDP
EVQFTWYINNEQVRTARPPLREQQFNSTIRVVSTLPIAHQDWLRGKEFK
CKVHNKALPAPIEKTISKARGQPLEPKVYTMGPPREELSSRSVSLTCMI
NGFYPSDISVEWEKNGKAEDNYKTTPAVLDSDGSYFLYSKLSVPTSEWQ
RGDVFTCSVMHEALHNHYTQKSISRSPGK;

the heavy chain comprises a heavy chain variable region and a heavy chain constant region comprising a sequence:

(SEQ ID NO: 4)
QSLEESGGRLVTPGTPLTLTCTVSGFSLSSYGVSWVRQAPGKGLEWIGI
INSDHSTYYAKWAKGRFTISKTSTTVDLKITSPTTEDTATYFCARSYGI
GSIFWGPGTLVTVSSGQPKAPSVFPLAPCCGDTPSSTVTLGCLVKGYLP
EPVTVTWNSGTLTNGVRTFPSVRQSSGLYSLSSVVSVTSSSQPVTCNVA
HPATNTKVDKTVAPSTCSKPTCPPPELLGGPSVFIFPPKPKDTLMISRT
PEVTCVVVDVSQDDPEVQFTWYINNEQVRTARPPLREQQFNSTIRVVST
LPIAHQDWLRGKEFKCKVHNKALPAPIEKTISKARGQPLEPKVYTMGPP
REELSSRSVSLTCMINGFYPSDISVEWEKNGKAEDNYKTTPAVLDSDGS
YFLYSKLSVPTSEWQRGDVFTCSVMHEALHNHYTQKSISRSPGK;

the heavy chain variable region comprises an amino acid terminal signal sequence, and optionally the heavy chain variable region amino acid terminal signal sequence comprises a sequence METGLRWLLLVAVLKGVQC (SEQ ID NO:5);
the heavy chain variable region having a signal sequence comprises the sequence

(SEQ ID NO: 6)
METGLRWLLLVAVLKGVQCQSLEESGGRLVTPGTPLTLTCTVSGFSLSS
YGVSWVRQAPGKGLEWIGIINSDHSTYYAKWAKGRFTISKTSTTVDLKI
TSPTTEDTATYFCARSYGIGSIFWGPGTLVTVSS,

or
the heavy chain comprising a variable and a constant domain and having a signal sequence comprises a sequence:

(SEQ ID NO: 7)
METGLRWLLLVAVLKGVQCQSLEESGGRLVTPGTPLTLTCTVSGFSLSS
YGVSWVRQAPGKGLEWIGIINSDHSTYYAKWAKGRFTISKTSTTVDLKI
TSPTTEDTATYFCARSYGIGSIFWGPGTLVTVSSGQPKAPSVFPLAPCC
GDTPSSTVTLGCLVKGYLPEPVTVTWNSGTLTNGVRTFPSVRQSSGLYS
LSSVVSVTSSSQPVTCNVAHPATNTKVDKTVAPSTCSKPTCPPPELLGG
PSVFIFPPKPKDTLMISRTPEVTCVVVDVSQDDPEVQFTWYINNEQVRT
ARPPLREQQFNSTIRVVSTLPIAHQDWLRGKEFKCKVHNKALPAPIEKT
ISKARGQPLEPKVYTMGPPREELSSRSVSLTCMINGFYPSDISVEWEKN
GKAEDNYKTTPAVLDSDGSYFLYSKLSVPTSEWQRGDVFTCSVMHEALH
NHYTQKSISRSPGK;

the light chain variable region (VL), if present, comprises:
(a) an amino acid sequence:

(SEQ ID NO: 2)
AAVLTQTPSPVSAAVGGTVSISCQSSKSVYNNNQLSWFQQKPGQRPKLL
IYEAFKLPSGVPSRFKGSGSGTQFTLTISDVQCDDAATYYCAAVYSDDS
DNSFGGGTEVVVK,

or

SEQ ID NO:2 having one or more amino acid substitutions, additions (insertions) or deletions, and the recombinant Ab, or Ag binding fragment thereof, or monomeric or dimeric ABP retains its ability to specifically bind to a human ICOS protein or polypeptide;
the light chain variable region (VL) one or more amino acid substitutions comprise one or more conservative amino acid substitutions, and optionally the one or more conservative amino acid substitutions comprise: a conservative substitution comprises: replacement of an aliphatic amino acid with another aliphatic amino acid; replacement of a serine with a threonine or vice versa; replacement of an acidic residue with another acidic residue; replacement of a residue bearing an amide group with another residue bearing an amide group; exchange of a basic residue with another basic residue; or replacement of an aromatic residue with another aromatic residue;
the light chain variable region further comprises at least a portion of a light chain constant region, and optionally the light chain constant region comprises an amino acid sequence:

(SEQ ID NO: 8)
GDPVAPTVLIFPPAADQVATGTVTIVCVANKYFPDVTVTWEVDGTTQTT
GIENSKTPQNSADCTYNLSSTLTLTSTQYNSHKEYTCKVTQGTTSVVQS
FNRGDC;

the light chain comprises a variable region and a constant region comprising the sequence:

(SEQ ID NO: 9)
AAVLTQTPSPVSAAVGGTVSISCQSSKSVYNNNQLSWFQQKPGQRPKLL
IYEAFKLPSGVPSRFKGSGSGTQFTLTISDVQCDDAATYYCAAVYSDDS
DNSFGGGTEVVVKGDPVAPTVLIFPPAADQVATGTVTIVCVANKYFPDV
TVTWEVDGTTQTTGIENSKTPQNSADCTYNLSSTLTLTSTQYNSHKEYT
CKVTQGTTSVVQSFNRGDC;

the light chain variable domain further comprises an amino terminal signal sequence, and optionally the light chain variable domain amino terminal signal sequence comprises a sequence MDTRAPTQLLGLLLLWLPGATF (SEQ ID NO: 10);
the light chain variable domain having a signal sequence comprises

(SEQ ID NO: 11)
MDTRAPTQLLGLLLLWLPGATFAAVLTQTPSPVSAAVGGTVSISCQSSK
SVYNNNQLSWFQQKPGQRPKLLIYEAFKLPSGVPSRFKGSGSGTQFTLT
ISDVQCDDAATYYCAAVYSDDSDNSFGGGTEVVVK,

or
the light chain having a signal sequence comprises

(SEQ ID NO: 12)
MDTRAPTQLLGLLLLWLPGATFAAVLTQTPSPVSAAVGGTVSISCQSSK
SVYNNNQLSWFQQKPGQRPKLLIYEAFKLPSGVPSRFKGSGSGTQFTLT
ISDVQCDDAATYYCAAVYSDDSDNSFGGGTEVVVKGDPVAPTVLIFPPA
ADQVATGTVTIVCVANKYFPDVTVTWEVDGTTQTTGIENSKTPQNSADC
TYNLSSTLTLTSTQYNSHKEYTCKVTQGTTSVVQSFNRGDC;

wherein SEQ ID NO:1, SEQ ID NO:2, SEQ ID NO:4, SEQ ID NO:6, SEQ ID NO: 7, SEQ ID NO:8, SEQ ID NO:9, SEQ ID NO:11, and/or SEQ ID NO: 12, has two, three, four, five, six, seven, eight, nine, ten, eleven, twelve thirteen, fourteen or fifteen conservative amino acid substitutions, and the recombinant Ab, or Ag binding fragment thereof, or monomeric or dimeric ABP retains its ability to specifically bind to a human ICOS protein or polypeptide;
the heavy chain constant region comprises amino acid sequence from a IgG, IgM, IgA, IgD or IgE isotype, or the light chain constant region comprises amino acid sequence from a kappa (κ) or lambda (λ) isotype;
the at least a portion of the heavy chain constant region, at least a portion of the light chain constant region, or at least a portion of the heavy chain constant region and the light chain constant region, is or comprises amino acid sequence of a human, a rabbit, a mouse or a rat origin or comprises constant region amino acid sequence derived from a human, a rabbit, a mouse or a rat;
wherein at least a portion of the heavy chain constant region, at least a portion of the light chain constant region, or at least a portion of the heavy chain constant region and the light chain constant region, is or comprises a synthetic amino acid sequence;
wherein the recombinant Ab, the Ag binding fragment thereof, or monomeric or dimeric ABP, or the heavy chain constant region, or the light chain constant region, or the heavy chain constant region and the light chain constant region, further comprises or is bound to a heterologous protein, peptide, or a compound or a composition, and optionally the heterologous protein or peptide, or the compound or a composition, comprises a detectable protein, a detectable agent or a binding moiety; and optionally the heterologous protein or peptide comprises a carrier protein, or the heterologous protein, peptide or the compound or composition, is covalently conjugated to the recombinant antibody (Ab), or Ag binding fragment thereof, or monomeric or dimeric ABP;
the detectable agent or binding moiety comprises a biotin, a fluorescent or chemiluminescent label, a fluorophore, perylene, fluorenyl, coumarin, 7-methoxycoumarin (Mca), 4-(dimethylaminoazo)benzene-4-carboxylic acid (dabcyl), Tamra, boron-dipyrromethene (BODIPY), or derivatives thereof, a dye, a radioisotope, a quantum dot or photoluminescent aqueous nanocrystal, a hapten, or an antibody binding epitope or domain, and optionally the dye is or comprises rhodamine, [2-(4-nitro-2,1,3-benzoxadiazol-7-yl)aminoethyl]trimethylammonium (NBD), nile red or nile blue, or is a fluorescent dye comprising sulfoindocyanine,, and optionally the fluorophore is or comprises a dansyl, a fluorescein, a carboxyfluorescein (FAM) or a 6-FAM moiety, and optionally the dye is or comprises a cyanine dye, a Cy3 or a Cy5, and optionally the hapten is or comprises a biotin, a theophylline, a digoxigenin, a carborane, a fluorescein or a bromodeoxyuridine moiety; and/or
the Ab, or Ag binding fragment thereof, or monomeric or dimeric ABP is a recombinant Ab, or Ag binding fragment thereof, or monomeric or dimeric ABP, or comprises a peptide or polypeptide made by a recombinant technique.

In alternative embodiments, provided are chimeric or recombinant nucleic acids comprising or consisting of: a nucleic acid sequence encoding a Ab, or Ag binding fragment thereof, or monomeric or dimeric ABP as provided herein.

In alternative embodiments of chimeric or recombinant nucleic acids as provided herein:
the chimeric or recombinant nucleic acid further comprises and is operatively linked to a transcriptional regulatory element, and optionally the transcriptional regulatory element comprises a promoter, and optionally the promoter is an inducible promoter or a constitutive promoter; and/or
the chimeric or recombinant nucleic acid further comprises sequence encoding an amino terminal signal peptide, and optionally the amino terminal signal peptide comprises the amino acid sequence: METGLRWLLLVAVLKGVQC (SEQ ID NO: 5); or MDTRAPTQLLGLLLLWLPGATF (SEQ ID NO: 10).

In alternative embodiments, provided are expression cassettes, vectors, recombinant viruses, artificial chromosomes, cosmids or plasmids comprising a chimeric or a recombinant nucleic acid as provided herein, including nucleic acids encoding antibodies (Ab), or antigen (Ag) binding fragments thereof, or monomeric or dimeric antigen binding proteins (ABP) as provided herein.

In alternative embodiments, provided are cells comprising, or having contained therein, a chimeric or recombinant antibody or dimeric antigen binding protein as provided herein, a chimeric or recombinant nucleic acid as provided herein, or an expression cassette, vector, recombinant virus, artificial chromosome, cosmid or plasmid as provided herein. In alternative embodiments of cells as provided herein: the cell is a bacterial, fungal, mammalian, yeast, insect or plant cell; or, the mammalian cell is a human cell.

In alternative embodiments, provided are methods for detecting the presence of a human ICOS (Inducible T-cell costimulatory) protein, or human CD278 protein or polypeptide, in or on a cell, a tissue, an organ or a portion of any of the foregoing, comprising:
(a) contacting the cell, tissue or organ or portion of any of the foregoing with at least one Ab, or Ag binding fragment thereof, or monomeric or dimeric ABP as provided herein, and
(b) detecting the specific binding of the at least one Ab, or Ag binding fragment thereof, or monomeric or dimeric ABP with a BCMA polypeptide, in or on the cell, tissue or organ or portion of any of the foregoing,
thereby detecting the presence of the human BCMA protein in or on the cell, tissue, organ or portion of any of the foregoing.

In alternative embodiments, of methods for detecting the presence of a human ICOS as provided herein:
the at least one Ab, or Ag binding fragment thereof, or monomeric or dimeric ABP comprises a variable heavy chain (VH) having an amino acid sequence comprising SEQ ID NO: 1 and a variable light chain (VL) having an amino acid sequence comprising SEQ ID NO:2;
the at least one Ab, or Ag binding fragment thereof, or monomeric or dimeric ABP comprises a heavy chain having an amino acid sequence comprising SEQ ID NO: 4 and a light chain having an amino acid sequence comprising SEQ ID NO:9;
the method comprises (or further comprises) contacting the cell, tissue or organ or portion of any of the foregoing with two Abs, or Ag binding fragments thereof, or monomeric or dimeric ABPs, or a mixture of two Abs, or Ag binding fragments thereof, or monomeric or dimeric ABPs, and optionally the contacting comprises use of an immunohistochemistry (IHC) assay;
the method further comprises contacting the Ab, or Ag binding fragment thereof, or monomeric or dimeric ABP, specifically bound to a human ICOS protein or human CD278 protein or polypeptide, with a detectable agent to indicate or signal the specific binding of the Ab, or Ag binding fragment thereof, or monomeric or dimeric ABP, to the human ICOS protein or human CD278 protein or polypeptide, and optionally the detectable agent specifically binds to the Ab, or Ag binding fragment thereof, or monomeric or dimeric ABP;
the cell, tissue, organ or a portion of any of the foregoing is or comprises: an early B cell, a pro-B cell, a pre-B lymphocyte, a mature B-lymphocyte, a follicular center cell, or a cell in a tonsil, an organ, a lymph node germinal center, a bone marrow stem cell, a myelopoietic cell, a lymphocyte, a parafollicular T lymphocyte, a subpopulation of parafollicular T lymphocytes, a liver bile canalicular cell, a renal glomerular cell, a proximal tubular cell, a breast myoepithelial cell, a stromal cell around or associated with an infiltrating tumor cell, a kidney cell, a cerebellum cell, a prostate cell, a pancreas cell, a bone marrow cell or an epithelial cell, and optionally the epithelial cell is a brain, lung, intestine, kidney, breast or placental epithelial cell, and optionally the organ is a liver, prostate, brain, pancreas, bladder, colon, esophagus, kidney, skin or lung, and optionally the cell, tissue, organ or a portion of any of the foregoing is or comprises a cancer or a tumour cell, or a carcinoma cell or a carcinoid tumor cell;
the cancer or tumour cell is or comprises or is derived from: a carcinoma cell (optionally a squamous carcinoma cell), a mamma carcinoma cell, a colon carcinoma or colorectal cancer cell, a melanoma cell (optionally a malignant melanoma cell) or a multiple myeloma cell, a plasmacytoma cell, a lymphoma cell, a bladder or urothelial cancer cell, a cervical cancer cell, an ovarian cancer cell, an esophageal cancer or an esophageal squamous cell; a malignant pleural mesothelioma cell, a prostate cancer cell, a cell from a microsatellite instability-high/deficient mismatch repair tumor, a human Papilloma Virus-positive or Epstein-Barr positive tumor cell, a hepatocellular carcinoma or liver cancer cell, a lung cancer (optionally a non-small cell lung cancer (NSCLC)) cell, a gastric cancer cell, a renal or kidney cancer cell, a pancreatic cancer cell, a breast cancer cell (optionally a triple-negative breast cancer cell), a lymphoma cell or a leukemia cell, or a mycosis fungoides cell; and/or
the lymphoma or leukemia cell is or is derived from: a B cell lymphoma, an acute lymphoblastic leukemia (ALL) cell, an angioimmunoblastic T-cell lymphoma cell, a cutaneous T-cell lymphoma, a chronic myelogenous leukemia in blast crisis, a diffuse large B-cell lymphoma cell, a hairy cell leukemia cell, a follicular lymphoma cell, a Burkitt's lymphoma, a diffuse large B-cell lymphoma or a mantle cell lymphoma, and optionally the B cell lymphoma is a Hodgkin's lymphoma or a non-Hodgkin's lymphoma, and optionally the non-Hodgkin's lymphoma is follicular lymphoma, a diffuse large B cell lymphoma, a marginal zone B cell lymphoma, a small lymphocytic lymphoma (SLL) or chronic lymphocytic leukemia, (CLL), a Burkitt lymphoma or a mantle cell lymphoma (MCL), and optionally the carcinoma cell is a head and neck squamous cell carcinoma (HNSCC) or a basal cell carcinoma (BCC) cell.

In alternative embodiments, provided are methods for detecting or diagnosing a cancer or a tumor, wherein the method comprises detecting expression or presence of a human a human ICOS (Inducible T-cell costimulatory) protein, or a human CD278 protein or polypeptide, in or on a cell, tissue or organ sample using a method as provided herein, and wherein the detecting of the specific binding of the Ab, or Ag binding fragment thereof, or monomeric or dimeric ABP with the human ICOS (Inducible T-cell costimulatory) protein, or the human CD278 protein or polypeptide in or on the cell, tissue or organ or portion of any of the foregoing, detects or diagnoses, or assists in the detection or diagnosis of, the cancer or the tumor.

In alternative embodiments of methods for detecting or diagnosing a cancer or a tumor:
the cancer or the tumor is or comprises or is derived from: a carcinoma cell (optionally a squamous carcinoma cell), or a mamma carcinoma cell, a colon carcinoma cell or colorectal cancer cell, a melanoma cell (optionally a malignant melanoma cell) or a multiple myeloma cell, a plasmacytoma cell, a lymphoma cell, a bladder or urothelial cancer cell, a cervical cancer cell, an ovarian cancer cell, an esophageal cancer or an esophageal squamous cell; a malignant pleural mesothelioma cell, a prostate cancer cell, a cell from a microsatellite instability-high/deficient mismatch repair tumor, a human Papilloma Virus-positive or Epstein-Barr positive tumor cell, a hepatocellular carcinoma or liver cancer cell, a lung cancer cell (optionally a non-small cell lung cancer (NSCLC) cell), a gastric cancer cell, a renal or kidney cancer cell, a pancreatic cancer cell, a breast cancer cell (optionally a triple-negative breast cancer cell), a lymphoma cell or a leukemia cell, or a mycosis fungoides cell;
the lymphoma or leukemia cell is or is derived from: a B cell lymphoma, an acute lymphoblastic leukemia (ALL) cell, an angioimmunoblastic T-cell lymphoma cell, a cutaneous T-cell lymphoma, a chronic myelogenous leukemia in blast crisis, a diffuse large B-cell lymphoma cell, a hairy cell leukemia cell, a follicular lymphoma cell, a Burkitt's lymphoma, a diffuse large B-cell lymphoma or a mantle cell lymphoma, and optionally the B cell lymphoma is a Hodgkin's lymphoma or a non-Hodgkin's lymphoma, and optionally the non-Hodgkin's lymphoma is follicular lymphoma, a diffuse large B cell lymphoma, a marginal zone B cell lymphoma, a small lymphocytic lymphoma (SLL) or chronic lymphocytic leukemia, (CLL), a Burkitt lymphoma or a mantle cell lymphoma (MCL), and optionally the carcinoma cell is a head and neck squamous cell carcinoma (HNSCC) or a basal cell carcinoma (BCC) cell;
the cell, tissue or organ sample is from an individual in need thereof or an individual at higher risk of having a cancer of a tumor, or having a family history of the cancer or tumor;
the detection comprises conducting an immunohistochemistry (IHC) assay, and optionally at least two Abs, or Ag binding fragments thereof, or monomeric or dimeric ABPs, are used to contact the cell, tissue or organ sample;
at least one of the two Abs, or Ag binding fragments thereof, or monomeric or dimeric ABPs comprises a variable heavy chain (VH) having an amino acid sequence comprising SEQ ID NO: 1 and a variable light chain (VL) having an amino acid sequence comprising SEQ ID NO:2; and/or
the at least one Ab, or Ag binding fragment thereof, or monomeric or dimeric ABP comprises a heavy chain having an amino acid sequence comprising SEQ ID NO: 4 and a light chain having an amino acid sequence comprising SEQ ID NO:9.

In alternative embodiments, provided are methods for treating, ameliorating or preventing a cancer or tumor comprising first detecting or diagnosing the cancer or tumor using a method as provided herein, followed by treatment of the individual in need thereof for the treatment, amelioration or prevention of the cancer or tumor.

In alternative embodiments of methods for treating, ameliorating or preventing a cancer or tumor as provided herein:
the cancer or the tumor is or comprises or is derived from: a carcinoma cell (optionally a squamous carcinoma cell), a mamma carcinoma cell, a colon carcinoma cell or colorectal cancer cell, a melanoma cell (optionally a malignant melanoma cell) or a multiple myeloma cell, a plasmacytoma cell, a lymphoma cell, a bladder or urothelial cancer cell, a cervical cancer cell, an ovarian cancer cell, an esophageal cancer or an esophageal squamous cell; a malignant pleural mesothelioma cell, a prostate cancer cell, a cell from a microsatellite instability-high/deficient mismatch repair tumor, a human Papilloma Virus-positive or Epstein-Barr positive tumor cell, a hepatocellular carcinoma or liver cancer cell, a lung cancer (optionally a non-small cell lung cancer (NSCLC)) cell, a gastric cancer cell, a renal or kidney cancer cell, a pancreatic cancer cell, a breast cancer cell (optionally a triple-negative breast cancer cell), a lymphoma cell or a leukemia cell, or a mycosis fungoides cell;
the lymphoma or leukemia cell is or is derived from: a B cell lymphoma, an acute lymphoblastic leukemia (ALL) cell, an angioimmunoblastic T-cell lymphoma cell, a cutaneous T-cell lymphoma, a chronic myelogenous leukemia in blast crisis, a diffuse large B-cell lymphoma cell, a hairy cell leukemia cell, a follicular lymphoma cell, a Burkitt's lymphoma, a diffuse large B-cell lymphoma or a mantle cell lymphoma, and optionally the B cell lymphoma is a Hodgkin's lymphoma or a non-Hodgkin's lymphoma, and optionally the non-Hodgkin's lymphoma is follicular lymphoma, a diffuse large B cell lymphoma, a marginal zone B cell lymphoma, a small lymphocytic lymphoma (SLL) or chronic lymphocytic leukemia, (CLL), a Burkitt lymphoma or a mantle cell lymphoma (MCL), and optionally the carcinoma cell is a head and neck squamous cell carcinoma (HNSCC) or a basal cell carcinoma (BCC) cell; and/or
the cell, tissue or organ sample is from an individual in need thereof, and optionally the individual in need thereof is an individual at higher risk of having a cancer of a tumor, or having a family history of the cancer or tumor.

In alternative embodiments, provided are uses of at least one recombinant antibody (Ab), or antigen (Ag) binding fragment thereof, or monomeric or dimeric antigen binding protein (ABP) as provided herein, or encoded by a nucleic acid as provided herein, for detecting or diagnosing a cancer, or treating, ameliorating or preventing a cancer.

In alternative embodiments of the uses as provided herein:
the cancer or the tumor is or comprises or is derived from: a carcinoma cell (optionally a squamous carcinoma cell), a mamma carcinoma cell, a colon carcinoma cell or colorectal cancer cell, a melanoma cell (optionally a malignant melanoma cell) or a multiple myeloma cell, a plasmacytoma cell, a lymphoma cell, a bladder or urothelial cancer cell, a cervical cancer cell, an ovarian cancer cell, an esophageal cancer or an esophageal squamous cell; a malignant pleural mesothelioma cell, a prostate cancer cell, a cell from a microsatellite instability-high/deficient mismatch repair tumor, a human Papilloma Virus-positive or Epstein-Barr positive tumor cell, a hepatocellular carcinoma or liver cancer cell, a lung cancer (optionally a non-small cell lung cancer (NSCLC)) cell, a gastric cancer cell, a renal or kidney cancer cell, a pancreatic cancer cell, a breast cancer cell (optionally a triple-negative breast cancer cell), a lymphoma cell or a leukemia cell, or a mycosis fungoides cell;
the lymphoma or leukemia cell is or is derived from: a B cell lymphoma, an acute lymphoblastic leukemia (ALL) cell, an angioimmunoblastic T-cell lymphoma cell, a cutaneous T-cell lymphoma, a chronic myelogenous leukemia in blast crisis, a diffuse large B-cell lymphoma cell, a hairy cell leukemia cell, a follicular lymphoma cell, a Burkitt's lymphoma, a diffuse large B-cell lymphoma or a mantle cell lymphoma, and optionally the B cell lymphoma is a Hodgkin's lymphoma or a non-Hodgkin's lymphoma, and optionally the non-Hodgkin's lymphoma is follicular lymphoma, a diffuse large B cell lymphoma, a marginal zone B cell lymphoma, a small lymphocytic lymphoma (SLL) or chronic lymphocytic leukemia, (CLL), a Burkitt lymphoma or a mantle cell lymphoma (MCL), and optionally the carcinoma cell is a head and neck squamous cell carcinoma (HNSCC) or a basal cell carcinoma (BCC) cell; and/or
the detection comprises conducting an immunohistochemistry (IHC) assay.

In alternative embodiments, provided are recombinant antibodies (Ab), or antigen (Ag) binding fragments thereof, or monomeric or dimeric antigen binding protein (ABP) as provided herein, for use in detecting or diagnosing a cancer, or treating, ameliorating or preventing a cancer. In alternative embodiments, wherein the cancer or the tumor is or comprises or is derived from: a carcinoma cell (optionally a squamous carcinoma cell), a mamma carcinoma cell, a colon carcinoma cell or colorectal cancer cell, a melanoma cell (optionally a malignant melanoma cell) or a multiple myeloma cell, a plasmacytoma cell, a lymphoma cell, a bladder or urothelial cancer cell, a cervical cancer cell, an ovarian cancer cell, an esophageal cancer or an esophageal squamous cell; a malignant pleural mesothelioma cell, a prostate cancer cell, a cell from a microsatellite instability-high/deficient mismatch repair tumor, a human Papilloma Virus-positive or Epstein-Barr positive tumor cell, a hepatocellular carcinoma or liver cancer cell, a lung cancer (optionally a non-small cell lung cancer (NSCLC)) cell, a gastric cancer cell, a renal or kidney cancer cell, a pancreatic cancer cell, a breast cancer cell (optionally a triple-negative breast cancer cell), a lymphoma cell or a leukemia cell, or a mycosis fungoides cell. In alternative embodiments, the lymphoma or leukemia cell is or is derived from: a B cell lymphoma, an acute lymphoblastic leukemia (ALL) cell, an angioimmunoblastic T-cell lymphoma cell, a cutaneous T-cell lymphoma, a chronic myelogenous leukemia in blast crisis, a diffuse large B-cell lymphoma cell, a hairy cell leukemia cell, a follicular lymphoma cell, a Burkitt's lymphoma, a diffuse large B-cell lymphoma or a mantle cell lymphoma, and optionally the B cell lymphoma is a Hodgkin's lymphoma or a non-Hodgkin's lymphoma, and optionally the non-Hodgkin's lymphoma is follicular lymphoma, a diffuse large B cell lymphoma, a marginal zone B cell lymphoma, a small lymphocytic lymphoma (SLL) or chronic lymphocytic leukemia, (CLL), a Burkitt lymphoma or a mantle cell lymphoma (MCL), and optionally the carcinoma cell is a head and neck squamous cell carcinoma (HNSCC) or a basal cell carcinoma (BCC) cell. In alternative embodiments the detecting or diagnosing comprises conducting an immunohistochemistry (IHC) assay,

In alternative embodiments, provided are kits comprising: a chimeric or recombinant antibody as provided herein; a chimeric or a recombinant nucleic acid as provided herein; or an expression cassette, vector, recombinant virus, artificial chromosome, cosmid or plasmid as provided herein; or, a cell as provided herein. In alternative embodiments of the kits as provided herein, the kit comprises components needed for an immunohistochemistry (IHC) assay, and/or comprises instructions for practicing a method as provided herein.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

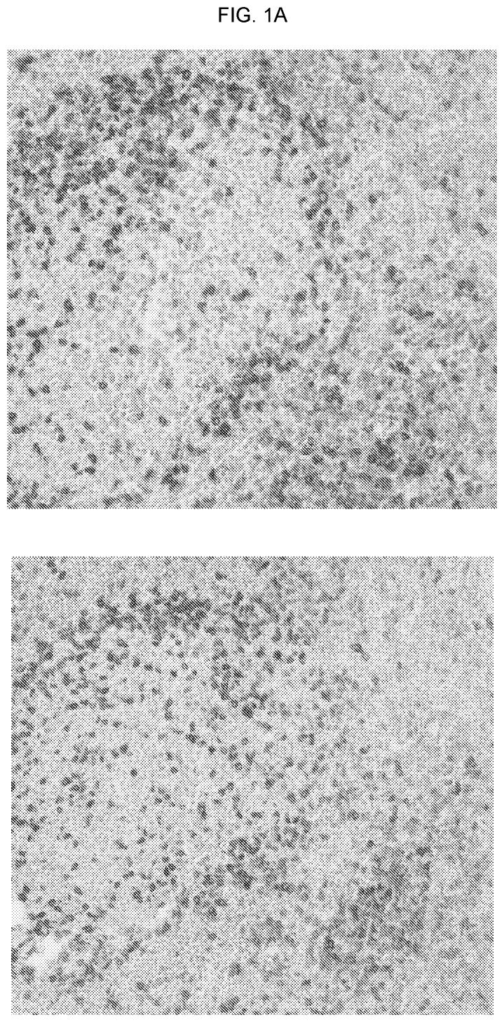

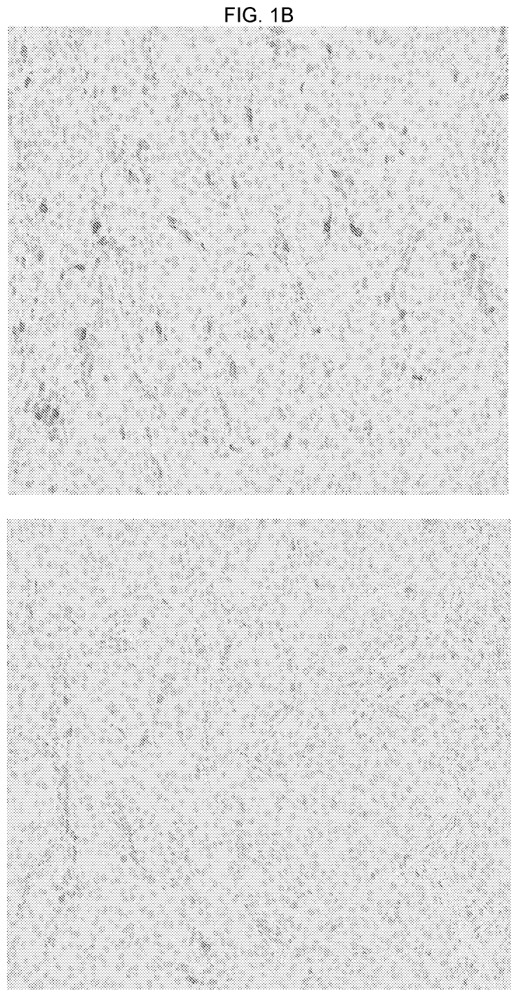

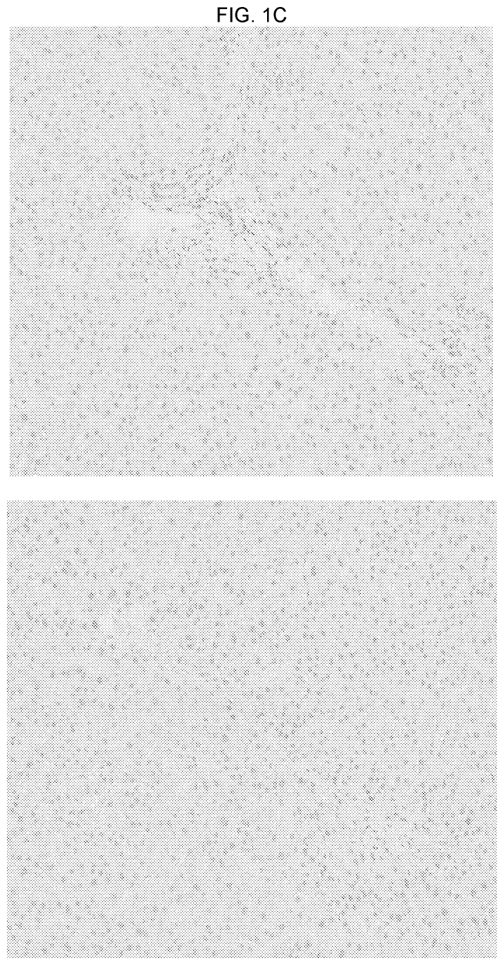

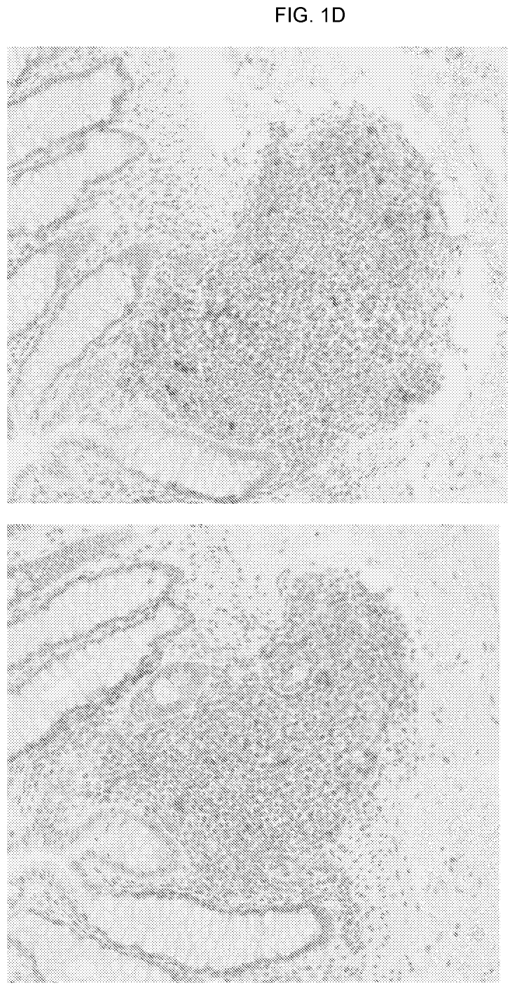

FIG. 1A-D illustrates images of: IHC comparing an exemplary anti-human human ICOS antibody (Ab) as provided herein having: a heavy chain having an amino acid sequence comprising SEQ ID NO:4; and, a light chain having an amino acid sequence comprising SEQ ID NO:9, the Ab also designated T0251A, with a reference anti-human human ICOS Ab (Abscam, designated SP98) in IHC staining of:
tonsil cells (FIG. 1A), T0251A upper image, SP98 lower image;
melanoma cells (FIG. 1B), T0251A upper image, SP98 lower image;
liver cells (FIG. 1C), T0251A upper image, SP98 lower image; and
colon cells (FIG. 1D), TO251A upper image, SP98 lower image.

Like reference symbols in the various drawings indicate like elements.

### DETAILED DESCRIPTION

In alternative embodiments, provided are chimeric, synthetic or recombinant anti-human ICOS protein (also called: inducible T-cell co-stimulator, or Cluster of Differentiation-278, or CD278; Activation-inducible lymphocyte immunomediatory molecule; AILIM; and, CVID1) binding polypeptides, including ICOS-binding antibodies (Abs), including products of manufacture and kits comprising them, and methods for making and using them, including for example their use in the detection or diagnosis, and treatment, of a cancer, or other diseases or conditions involving expression of ICOS.

While the invention is not limited by any particular mechanism of action, in alternative embodiments antibodies or antigen binding proteins as provided herein target (and specifically bind to) ICOS protein expressed on the surface of cancer cells, as seen in some types of cancer such as a carcinoma (optionally a squamous carcinoma), a mamma carcinoma, a colon carcinoma or a colorectal cancer, a melanoma (optionally a malignant melanoma) or a multiple myeloma, a plasmacytoma, a lymphoma, a bladder or urothelial cancer, a cervical cancer, an ovarian cancer, an esophageal cancer or an esophageal squamous; a malignant pleural mesothelioma, a prostate cancer, a microsatellite instability-high/deficient mismatch repair tumor, a human Papilloma Virus-positive or Epstein-Barr positive tumor, a hepatocellular carcinoma or liver cancer, a lung cancer (optionally a non-small cell lung cancer (NSCLC)), a gastric cancer, a renal or kidney cancer, a pancreatic cancer, a breast cancer (optionally a triple-negative breast cancer), a lymphoma or a leukemia, or a mycosis fungoides.

### Expression of Recombinant or Chimeric Antibodies

In alternative embodiments, chimeric or recombinant Abs as provided herein, including the exemplary chimeric or recombinant anti-human ICOS Ab, with signal peptide, or without the signal peptide, can be expressed as a recombinant Ab using a plasmid (or any expression vehicle) encoding the respective heavy and light chains, or the heavy chain and the light chain can be encoded in separate expression vehicles.

In some embodiments, the heavy and light chains can be (cis- or trans-) expressed from a pTT5™ vector(s) (National Research Council Canada, NRC-CNRC, Canada) in HEK293-6E cells. In alternative embodiment, the vector or vectors expressing the heavy and/or light chains are episomal or are chromosomally integrated, for example, in a stable cell line capable of synthesizing, optionally inducibly synthesizing, the heavy and/or light chains.

In alternative embodiments, provided are nucleic acids encoding chimeric or recombinant Abs as provided herein. Nucleic acids as provided herein can be made, isolated and/or manipulated by, for example, cloning and expression of cDNA libraries, amplification of message or genomic DNA by PCR, and the like. Nucleic acids used to practice embodiments as provided herein, whether RNA, cDNA, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, fungal, mammalian, yeast, insect or plant cell expression systems.

Alternatively, these nucleic acids can be synthesized in vitro by well-known chemical synthesis techniques, as described in, for example, Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, for example, subcloning, labeling probes (for example, random-primer labeling using Klenow polymerase, nick translation, amplification), sequencing, hybridization and the like are well described in the scientific and patent literature, see, for example, Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used to practice embodiments as provided herein comprises screening and re-cloning inserts isolated or amplified from, for example, genomic clones or cDNA clones. Sources of nucleic acids include recombinant nucleic acid sequences, genomic or cDNA libraries contained and/or expressed in, for example, mammalian artificial chromosomes (MACs), see, for example, U.S. Pat. Nos. 5,721,118; 6,025,155; human artificial chromosomes, see, for example, Rosenfeld (1997) Nat. Genet. 15:333-335; yeast artificial chromosomes (YAC); bacterial artificial chromosomes (BAC); P1 artificial chromosomes, see, for example, Woon (1998) Genomics 50:306-316; P1-derived vectors (PACs), see, for example, Kern (1997) Biotechniques 23:120-124; cosmids, recombinant viruses, phages, phagemids or plasmids.

In alternative embodiments, nucleic acids as provided herein are operably linked to transcriptional regulatory elements, including promoters, with can be constitutive or inducible transcriptional regulatory elements.

In alternative aspects, provided are “expression cassettes” comprising a nucleotide sequence as provided herein, for example encoding a chimeric or recombinant antibody as provided herein. Expression cassettes can include at least a transcriptional regulatory element, for example, a promoter, operably linked with an antibody coding sequence, and optionally can also include transcription termination signals. Additional factors necessary or helpful in effecting expression may also be used, for example, enhancers.

In alternative aspects, expression cassettes used to practice embodiments as provided herein include plasmids, expression vectors, recombinant viruses, any form of recombinant “naked DNA” vector, and the like. In alternative aspects, a “vector” used to practice embodiments as provided herein can comprise a nucleic acid that can infect, transfect, transiently or permanently transduce a cell. In alternative aspects, a vector used to practice embodiments as provided herein can be a naked nucleic acid, or a nucleic acid complexed with protein or lipid. In alternative aspects, vectors used to practice embodiments as provided herein can comprise viral or bacterial nucleic acids and/or proteins, and/or membranes (for example, a cell membrane, a viral lipid envelope, etc.). In alternative aspects, vectors used to practice embodiments as provided herein can include, but are not limited to replicons (for example, RNA replicons, bacteriophages) to which fragments of DNA may be attached and become replicated. Vectors thus include, but are not limited to RNA, autonomous self-replicating circular or linear DNA or RNA (for example, plasmids, viruses, and the like, see, for example, U.S. Pat. No. 5,217,879), and can include both the expression and non-expression plasmids. In alternative aspects, the vector used to practice embodiments as provided herein can be stably replicated by the cells during mitosis as an autonomous structure, or can be incorporated within the host's genome.

In alternative aspects, “promoters” used to practice embodiments as provided herein include all sequences capable of driving transcription of a coding sequence in a cell, for example, a bacterial, yeast, fungal, plant, insect (for example, baculovirus) or mammalian cell. Thus, promoters used in the constructs include cis-acting transcriptional control elements and regulatory sequences that are involved in regulating or modulating the timing and/or rate of transcription of a gene. For example, a promoter used to practice embodiments as provided herein can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences can interact with proteins or other biomolecules to carry out (turn on/off, regulate, modulate, etc.) transcription.

“Constitutive” promoters used to practice embodiments as provided herein can be those that drive expression continuously under most environmental conditions and states of development or cell differentiation. “Inducible” or “regulatable” promoters used to practice embodiments as provided herein can direct expression of a nucleic acid as provided herein under the influence of environmental conditions or developmental conditions. Examples of environmental conditions that may affect transcription by inducible promoters used to practice embodiments as provided herein include the presence of an inducing factor administered to a cell.

In alternative embodiments, nucleic acids used to practice embodiments as provided herein encode polypeptides having the following amino acid sequences:

### Summary Exemplary Polypeptide Sequences

(SEQ ID NO:1) Variable domain of IgG heavy chain
(SEQ ID NO:2) Variable domain of kappa1 light chain
(SEQ ID NO:3) heavy chain constant region:
(SEQ ID NO:4) heavy chain variable and constant regions:
(SEQ ID NO:5) heavy chain variable region amino acid terminal signal sequence
(SEQ ID NO:6) heavy chain variable region having a signal sequence
(SEQ ID NO:7) heavy chain with variable and constant domain with amino terminal signal sequence
(SEQ ID NO:8) light chain constant region
(SEQ ID NO:9) light chain with variable region and a constant region
(SEQ ID NO:10) light chain variable domain amino terminal signal sequence
(SEQ ID NO:11) light chain variable domain having a signal sequence
(SEQ ID NO:12) light chain having a signal sequence

### Exemplary Polypeptide Sequences

SEQ ID NO: 1 (Variable domain of IgG heavy chain)
QSLEESGGRLVTPGTPLTLTCTVSGFSLSSYGVSWVRQAPGKGLEWIGIINSDHSTYYAKWAKGRFTISK
TSTTVDLKITSPTTEDTATYFCARSYGIGSIFWGPGTLVTVSS
SEQ ID NO: 2 (Variable domain of kappa1 light chain)
AAVLTQTPSPVSAAVGGTVSISCQSSKSVYNNNQLSWFQQKPGQRPKLLIYEAFKLPSGVPSRFKGSGSG
TQFTLTISDVQCDDAATYYCAAVYSDDSDNSFGGGTEVVVK
(SEQ ID NO: 3) heavy chain constant region:
GQPKAPSVFPLAPCCGDTPSSTVTLGCLVKGYLPEPVTVTWNSGTLTNGVRTFPSVRQSSGLYSLSSVVS
VTSSSQPVTCNVAHPATNTKVDKTVAPSTCSKPTCPPPELLGGPSVFIFPPKPKDTLMISRTPEVTCVVVD
VSQDDPEVQFTWYINNEQVRTARPPLREQQFNSTIRVVSTLPIAHQDWLRGKEFKCKVHNKALPAPIEK
TISKARGQPLEPKVYTMGPPREELSSRSVSLTCMINGFYPSDISVEWEKNGKAEDNYKTTPAVLDSDGSY
FLYSKLSVPTSEWQRGDVFTCSVMHEALHNHYTQKSISRSPGK
(SEQ ID NO: 4) heavy chain variable and constant regions:
QSLEESGGRLVTPGTPLTLTCTVSGFSLSSYGVSWVRQAPGKGLEWIGIINSDHSTYYAKWAKGRFTISK
TSTTVDLKITSPTTEDTATYFCARSYGIGSIFWGPGTLVTVSSGQPKAPSVFPLAPCCGDTPSSTVTLGCL
VKGYLPEPVTVTWNSGTLTNGVRTFPSVRQSSGLYSLSSVVSVTSSSQPVTCNVAHPATNTKVDKTVAP
STCSKPTCPPPELLGGPSVFIFPPKPKDTLMISRTPEVTCVVVDVSQDDPEVQFTWYINNEQVRTARPPL
REQQFNSTIRVVSTLPIAHQDWLRGKEFKCKVHNKALPAPIEKTISKARGQPLEPKVYTMGPPREELSSR
SVSLTCMINGFYPSDISVEWEKNGKAEDNYKTTPAVLDSDGSYFLYSKLSVPTSEWQRGDVFTCSVMHE
ALHNHYTQKSISRSPGK
(SEQ ID NO: 5) heavy chain variable region amino acid terminal signal sequence
METGLRWLLLVAVLKGVQC
(SEQ ID NO: 6) heavy chain variable region having a signal sequence
METGLRWLLLVAVLKGVQCQSLEESGGRLVTPGTPLTLTCTVSGFSLSSYGVSWVRQAPGKGLEWIGII
NSDHSTYYAKWAKGRFTISKTSTTVDLKITSPTTEDTATYFCARSYGIGSIFWGPGTLVTVSS
heavy chain with variable and constant domain with amino terminal signal sequence
(SEQ ID NO: 7)
METGLRWLLLVAVLKGVQCQSLEESGGRLVTPGTPLTLTCTVSGFSLSSYGVSWVRQAPGKGLEWIGII
NSDHSTYYAKWAKGRFTISKTSTTVDLKITSPTTEDTATYFCARSYGIGSIFWGPGTLVTVSSGQPKAPS
VFPLAPCCGDTPSSTVTLGCLVKGYLPEPVTVTWNSGTLTNGVRTFPSVRQSSGLYSLSSVVSVTSSSQP
VTCNVAHPATNTKVDKTVAPSTCSKPTCPPPELLGGPSVFIFPPKPKDTLMISRTPEVTCVVVDVSQDDP
EVQFTWYINNEQVRTARPPLREQQFNSTIRVVSTLPIAHQDWLRGKEFKCKVHNKALPAPIEKTISKARG
QPLEPKVYTMGPPREELSSRSVSLTCMINGFYPSDISVEWEKNGKAEDNYKTTPAVLDSDGSYFLYSKLS
VPTSEWQRGDVFTCSVMHEALHNHYTQKSISRSPGK
light chain constant region:
(SEQ ID NO: 8)
GDPVAPTVLIFPPAADQVATGTVTIVCVANKYFPDVTVTWEVDGTTQTTGIENSKTPQNSADCTYNLSS
TLTLTSTQYNSHKEYTCKVTQGTTSVVQSFNRGDC
light chain with variable region and a constant region:
(SEQ ID NO: 9)
AAVLTQTPSPVSAAVGGTVSISCQSSKSVYNNNQLSWFQQKPGQRPKLLIYEAFKLPSGVPSRFKGSGSG
TQFTLTISDVQCDDAATYYCAAVYSDDSDNSFGGGTEVVVKGDPVAPTVLIFPPAADQVATGTVTIVCV
ANKYFPDVTVTWEVDGTTQTTGIENSKTPQNSADCTYNLSSTLTLTSTQYNSHKEYTCKVTQGTTSVV
QSFNRGDC
(SEQ ID NO: 10) light chain variable domain amino terminal signal sequence
MDTRAPTQLLGLLLLWLPGATF
(SEQ ID NO: 11) light chain variable domain having a signal sequence comprises
MDTRAPTQLLGLLLLWLPGATFAAVLTQTPSPVSAAVGGTVSISCQSSKSVYNNNQLSWFQQKPGQRPK
LLIYEAFKLPSGVPSRFKGSGSGTQFTLTISDVQCDDAATYYCAAVYSDDSDNSFGGGTEVVVK
(SEQ ID NO: 12) light chain having a signal sequence comprises
MDTRAPTQLLGLLLLWLPGATFAAVLTQTPSPVSAAVGGTVSISCQSSKSVYNNNQLSWFQQKPGQRPK
LLIYEAFKLPSGVPSRFKGSGSGTQFTLTISDVQCDDAATYYCAAVYSDDSDNSFGGGTEVVVKGDPVAP
TVLIFPPAADQVATGTVTIVCVANKYFPDVTVTWEVDGTTQTTGIENSKTPQNSADCTYNLSSTLTLTST
QYNSHKEYTCKVTQGTTSVVQSFNRGDC

### Peptides, Polypeptides, Peptidomimetics

In alternative embodiments, peptides and polypeptides used to practice embodiments as provided herein can comprise any “mimetic” and/or “peptidomimetic” form. In alternative embodiments, peptides and polypeptides used to practice embodiments as provided herein can comprise synthetic chemical compounds which have substantially the same structural and/or functional characteristics of the natural polypeptide, for example, a chimeric or recombinant antibody as provided herein. The mimetic used to practice embodiments as provided herein can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. Routine experimentation will determine whether a mimetic is effective for practicing the invention, for example, if a mimetic composition is effective in specifically binding ICOS protein. Methodologies detailed herein and others known to persons skilled in the art may be used to select or guide one to choose effective mimetic for practicing the compositions and/or methods of this invention.

Polypeptide mimetic compositions for practicing embodiments as provided herein can comprise any combination of non-natural structural components. In alternative aspects, mimetic compositions for practicing embodiments as provided herein can comprise one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, for example, a beta turn, gamma turn, beta sheet, alpha helix conformation, and the like. For example, a polypeptide can be characterized as a mimetic when all or some of its residues are joined by chemical means other than natural peptide bonds. In alternative aspects, mimetic compositions for practicing embodiments as provided herein can be peptoids, where the side chain is connected to the nitrogen of the peptide backbone, instead of the α-carbon as in peptides; and, peptoids lack the amide hydrogen which is responsible for many of the secondary structure elements in peptides and proteins. In alternative aspects, mimetic compositions for practicing embodiments as provided herein can be beta peptides (β-peptides), in which the amino group is attached to the β-carbon (i.e. the carbon two atoms away from the carboxylate group).

In alternative embodiments, peptides and polypeptides used to practice embodiments as provided herein can be purposely deuterated, for example, a hydrogen is replaced by a deuterium (“-D”) a particular position, and it is understood that the abundance of deuterium at that position is greater than, or substantially greater than, the natural abundance of deuterium, which is 0.015%. For example, in alternative embodiments deuterium substitution, or “enrichment”, occurs at a specific position or positions. In one embodiment, the deuterium enrichment is no less than about 1%, or the deuterium enrichment is no less than about 1%, 5%, 10%, 20%, 50%, 70%, 80% or 90%, or is between about 1% and 99%.

### Exemplary Immunohistochemistry (IHC) Techniques and Armamentarium

In alternative embodiments, immunohistochemistry (IHC) methodologies and/or reagents used to practice compositions, products of manufacture, kits or methods as provided herein can include or comprise or comprise use of any IHC protocol, IHC armamentarium, devices and/or image or data analysis system, for practicing IHC or IHC reagents known in the art, for example, as described in U.S. patent nos. (USPNs) 11,321,881 (describing IHC imaging protocols and apparatus); 11,249,085, describing applications of click chemistry for signal amplification in IHC and ISH assays; 11,222,424 (describing IHC imaging apparatus and computer programs); 11,143,648, describing new colors for chromogenic IHC and ISH staining with multi-dye quinone methide and tyramide conjugates; 11,047,774 (describing automated IHC specimen processing systems); 11,028,044, describing processes for commercial scale preparation of 3,3′5,5′-tetramethylbenzidine (TMB) and its salts; 10,977,791, describing computer-implemented methods for analysis of a tissue sample; 10,948,493, describing IHC methods; 10,937,162 (describing IHC image analysis algorithms); 10,816,443, describing automated batch stainers for staining biological specimens on microcope slides; 10,718,773, describing methods for determining the eligibility of a subject having a malignancy for treatment with a therapeutic agent; 10,565,479 (describing methods for identifying blurred areas in digital images of stained tissue); 10,564,076 (describing systems for analytical (or IHC) sample preparation); 10,551,395 (describing an automated histological staining system); 10,551,378 (describing a tissue staining method); 10,504,224 (describing a digital tissue image analysis system for IHC); 10,501,777 (describing simultaneous, multiplexed detection and quantification of protein expression in IHC); 10,488,340 (describing method for extracting an image of a target fluorophore in a biological material); 10,453,195 (describing methods of detecting tissue areas of interest using digital pathology imaging); 10,438,381 (describing devices, systems and methods for generating a digital image of a tissue section); 10,416,176 (describing methods for processing specimens in an automated histological staining system); 10,393,633 (describing methods for processing and inhibiting the degradation of an IHC sample); 10,217,011 (describing handling of IHC slides); 10,209,165 (describing automated or semi-automated methods for assessing the quality of staining of a specimen containing cells); 10,126,216 (describing methods for fixing tissue samples for IHC); U.S. Pat. Nos. 9,423,322; 9,103,822, describing polymeric carriers for immunohistochemistry and in situ hybridization; 8,515,683 (describing methods and systems for automated detection of immunohistochemical (IHC) patterns); U.S. Pat. No. 10,816,443 (describing automated batch stainers for staining biological specimens on microscope slides); or as described in U.S. patent application publication nos. US 2021/0239683A1, describing compositions for forming a porous hydrogel around a cell suitable for immunostaining of cells within the hydrogel; or U.S. Pat. No. 11,112,413, or US 2022/0057408 A1, describing methods of employing the epitope-tagged antibodies for detecting one or more targets in an IHC tissue sample; or US 2021/0048432 A1, describing direct immunohistochemical (IHC) staining and direct immunocytochemistry (ICC) techniques; or US 2020/0292536, describing synthetic controls useful in immunohistochemistry (IHC) assays; or US 2022/0057408 A1, describing use of antibodies with epitope tags in immunohistochemistry (IHC) assays; or US 2021/0201485 A1, describing computer-implemented methods for analysis of a tissue sample; US 2019/0178867 A1 (describing detection of specific tissue objects within thin sections of tissue samples as imaged in a brightfield microscope without using a chromogenic stain that is specific to those tissue objects); US 2019/0156510 A1 (describing an image analysis method for analyzing an IHC tissue sample); US 2019/0293637 A1 (methods and systems for quantitative immunohistochemistry (IHC) of a target protein molecule); US 2019/0080450 A1 (describing an automated determination of the staining quality of an IHC stained biological sample); or, US 2020/0316589 A1 (describing a multi-well solid support vessel for the processing and testing of fixed biological materials); or US 2022/0229062 A1, describing methods or producing rapid immunohistochemical detection of an antigen from a biological sample; US 2021/0371520 A1, describing various immuno-histochemistry (IHC) methodologies;, or, US 2022/0034766 A1, describing immunohistochemical staining techniques to identify cells.

In alternative embodiments, chimeric or the recombinant antibodies, antigen binding fragments thereof, or monomeric or dimeric antigen binding proteins, in IHC protocols, or kits, as provided herein are substantially purified or isolated or are in the form of an unpurified or partially purified culture supernatant.

In alternative embodiments, methods as provided herein can use or comprise reagents for detecting or visualizing an antibody-antigen interaction using any products or methods know in the art, for example, and IHC protocol or reagents.

In alternative embodiments, methods as provided herein comprise use of chromogenic immunohistochemistry (CIH), wherein a primary antibody (for example, chimeric or a recombinant antibodies (Ab), or antigen binding fragments thereof, or monomeric or dimeric antigen binding proteins as provided herein) or secondary antibody (for example, where the secondary antibody binds to (the primary antibody) chimeric or a recombinant antibodies (Ab), or antigen binding fragments thereof, or monomeric or dimeric antigen binding proteins as provided herein after they have specifically bound to, paired with or associated with, an ICOS epitope or polypeptide) is conjugated to an enzyme, such as peroxidase (or immunoperoxidase), for example, a horseradish peroxidase (HRP), that can catalyze a color-producing reaction.

In alternative embodiments, methods as provided herein comprise use of immunofluorescence, where a primary or a secondary antibody is tagged to a fluorophore, such as fluorescein or fluorescein isothiocyanate (FITC), a triarylmethane dye such as rhodamine or rhodamine derivatives (for example, tetramethylrhodamine (TRITC), rhodamine 6G, rhodamine 123, rhodamine B, carboxytetramethylrhodamine (TAMRA), tetramethylrhodamine (TMR), sulforhodamine 101), aminomethylcoumarin acetate (AMCA), ALEXA™ or DYLIGHT™ fluors. 3,3′-Diaminobenzidine (DAB) also can be used.

In alternative embodiments, methods as provided herein comprise use of a direct method or one-step staining method where a primary antibody (for example, chimeric or a recombinant antibodies (Ab), or antigen binding fragments thereof, or monomeric or dimeric antigen binding proteins as provided herein) is labeled and reacts directly with an antigen, for example, in a tissue sections. While this technique utilizes only one antibody and therefore is simple and rapid, the sensitivity may be lower due to little signal amplification.

In alternative embodiments, methods as provided herein comprise use of an indirect method where an unlabeled primary antibody (first layer) binds to a target antigen (for example, TTF-1), for example, in a tissue or organ, and a labeled secondary antibody (second layer) then is reacted with the primary antibody. The secondary antibody can be against the isotype, for example, IgG, of the animal species in which the primary antibody is derived. This method can be more sensitive than direct detection strategies because of signal amplification due to the binding of several secondary antibodies to each primary antibody if the secondary antibody is conjugated to a detecting agent such as a fluorescent or enzyme reporter.

In alternative embodiments, further amplification is achieved if the secondary antibody is conjugated to several detecting molecules, for example, biotin molecules, which can recruit complexes of avidin-, streptavidin- or NEUTRAVIDIN™ protein-bound enzyme.

In alternative embodiments, the IHC is performed on tissue sections or tissue biopsies, for example, paraformaldehyde (PFA) fixed tissues or organs, or formalin-fixed paraffin-embedded tissues. In alternative embodiments, a tissue is sliced or used whole. Before sectioning, the tissue sample can be embedded in a medium, for example, paraffin wax or cryomedia. Tissue sections can be sliced on a variety of instruments, most commonly using a microtome, cryostat, or vibratome. Specimens can be sliced at a range of about 3 μm to 5 μm. The slices can be mounted on slides, dehydrated using alcohol washes of increasing concentrations (for example, 50%, 75%, 90%, 95%, 100%), and cleared using a detergent like xylene before being imaged under a microscope.

Depending on the method of fixation and tissue preservation, the sample may require additional steps to make the ICOS epitopes available for antibody binding, including deparaffinization and antigen retrieval. For formalin-fixed paraffin-embedded tissues, antigen-retrieval is often necessary, and can comprise pre-treating the sections with heat or proteases.

In alternative embodiments, the IHC is performed using an ENVISION DUOFLEX DOUBLESTAIN SYSTEM™ (En Vision DuoFLEX Doublestain System) (Agilent, San Jose, CA), which allows for staining of two or more markers on a single slide. In alternative embodiments, the IHC is performed using an En Vision FLEX HRP Magenta, High pH (DAKO OMNIS™, Agilent, San Jose, CA) system, and binding can be visualized by En Vision FLEX HRP™ Magenta Chromogen. In alternative embodiments, the IHC is performed using En Vision FLEX Mini Kit™, High pH, which is a high-sensitivity visualization system intended for use in IHC together with DAKO AUTOSTAINER™ instruments; this dual link system detects primary mouse and rabbit antibodies and the reaction is visualized by 3,3′-Diaminobenzidine (DAB) chromogen (DAB forms a water-insoluble brown precipitate when oxidized, for example, by a peroxidase).

### Products of Manufacture and Kits

Provided are products of manufacture and kits for practicing methods as provided herein, for example, comprising chimeric or recombinant anti-ICOS binding proteins such as anti-ICOS polypeptide Abs as provided herein; and optionally the products of manufacture and kits can further comprise some or all reagents needed to perform an IHC, and optionally can comprise instructions for practicing methods as provided herein.

In alternative embodiments, the products of manufacture, or kits, comprise mixtures or cocktails of antibodies (Abs) as provided herein, for example, a mixture or cocktail comprising two, three or more anti-human ICOS binding proteins such as anti-ICOS antibodies (Abs).

In alternative embodiments, the products of manufacture, or kits, comprise mixtures or cocktails of antibodies (Abs) comprising antibodies comprising heavy chain and/or light chain CDRs of antibodies as provided herein, or as produced by antibody-producing clones as provided herein.

In alternative embodiments, the products of manufacture, or kits, comprise antibody-producing clones as provided herein.

Any of the above aspects and embodiments can be combined with any other aspect or embodiment as disclosed here in the Summary, FIGURES and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR-Basics: From Background to Bench, First Edition, Springer Verlag, Germany.

### Example 1: Making Exemplary Abs

ICOS antigen was designed using part of the intracellular domain of human ICOS. The antigen was produced as a synthetic peptide. This antigen was used for immunizations of rabbits, bleeds were later taken to confirm serum reactivity against human ICOS in ELISA and IHC.

The rabbits showing best immune response against human ICOS, as tested on multiple ICOS positive human tissues and different non-expressing tissues were chosen for B-cell selection using blood samples from the immunized rabbits.

Briefly, B-cells expressing antibodies binding the immunogen were isolated as monoclonals and cultured before testing for ICOS specificity in ELISA. ELISA specific clones were further tested in super sensitive IHC on normal and clinical tissues, using high pH antigen retrieval buffers. The best performing clones were chosen based on IHC performance. The antibody variable domains were cloned into a custom-made expression vector based on the pTT5™ (National Research Council Canada, NRC-CNRC, Canada) backbone, containing the constant domains of the heavy and kappa1 light chain, respectively. Recombinant antibodies were expressed in HEK293-6E cells.

The recombinant antibodies were tested for human ICOS binding by biolayer interferometry (BLI) on a BLItz, and subsequently tested in IHC by standard FLEX protocols on normal and clinical tissues.

Some antibodies were identified showing ICOS specific staining in IHC. Clone 1E9/4B7 showed a very promising result on tested tissues. The antibody has been tested for specificity on different clinical tissues showing usefulness for in vitro diagnostics by immunohistochemistry.

FIG. 1A-D illustrates images of: an exemplary anti-human human ICOS antibody (Ab) as provided herein having: a heavy chain having an amino acid sequence comprising SEQ ID NO:4; and, a light chain having an amino acid sequence comprising SEQ ID NO:9, also designated T0251A, was compared to a reference anti-human human ICOS Ab (Abscam, designated SP98) in IHC staining of tonsil cells (FIG. 1A), melanoma cells (FIG. 1B), liver cells (FIG. 1C) and colon cells (FIG. 1D). A DAKO OMNIS™ (Agilent, San Jose, CA) staining IHC protocol was used for both the T0251A and the SP98 IHC staining. T0251A was used at 0.5 μg/mL, and SP98 used at 1/50 dilution, both T0251A and SP98 IHCs using TARGET RETRIEVAL SOLUTION™ (TRS) (Agilent, San Jose, CA) high pH, with rabbit linker.

A number of embodiments of the invention have been described. Nevertheless, it can be understood that various modifications may be made without departing from the spirit and scope of the invention. Accordingly, other embodiments are within the scope of the following claims.

1. An isolated or purified antibody (Ab), or antigen (Ag) binding fragment thereof, or monomeric or dimeric antigen binding protein (ABP), capable of specifically binding a human ICOS (Inducible T-cell costimulatory) protein or polypeptide, wherein the isolated or purified Ab, or Ag binding fragment thereof, or monomeric or dimeric ABP comprises:
(a) a heavy chain variable region (VH) comprising
an amino acid sequence comprising the three CDR1, CDR2 and CDR3 complementarity determining regions (CDRs) of SEQ ID NO: 1, or CDR1 amino acid (aa) residues GFSLSSYG (residues 25-32 of SEQ ID NO: 1), CDR2 aa residues INSDHST (residues 50 to 56 of SEQ ID NO: 1), and CDR3 aa residues ARSYGIGSIF (residues 93-102 of SEQ ID NO: 1),
and
(b) a light chain variable region (VL) comprising
an amino acid sequence comprising the three CDR1, CDR2 and CDR3 complementarity determining regions (CDRs) of SEQ ID NO:2, or CDR1 amino acid (aa) residues KSVYNNNQ (residues 27-34 of SEQ ID NO:2), CDR2 aa residues EAF (residues 52 to 54 of SEQ ID NO:2), and CDR3 aa residues AAVYSDDSDNS (residues 91-101 of SEQ ID NO:2).

2. The isolated or purified Ab, or Ag binding fragment thereof, or monomeric or dimeric ABP, of claim 1, further defined as a Fab, a F(ab′)2, a Fab′, a single-chain variable fragment (scFv), a (SCFV)2, a di-scFv, a bi-scFv, a minibody, a diabody, a triabody, a tetrabody, a single-domain antibody (dAB), a plurality of complementarity determining region (CDR) fragments, or a multispecific antibody formed from two or more antibody fragments.

3. The isolated or purified Ab, or Ag binding fragment thereof, or monomeric or dimeric ABP, of claim 1, wherein the heavy chain variable region (VH) comprises an amino acid sequence at least 70% identical to the amino acid sequence off
SEQ ID NO: 1.

4. (canceled)

5. (canceled)

6. The isolated or purified Ab, or Ag binding fragment thereof, or monomeric or dimeric ABP, of claim 1, wherein the heavy chain variable region (VH) further comprises at least a portion of a heavy chain constant region.

7. The isolated or purified Ab, or Ag binding fragment thereof, or monomeric or dimeric ABP, of claim 6, wherein the heavy chain constant region comprises an amino acid sequence at least 70% identical to the amino acid sequence of SEQ ID NO:3.

8-11. (canceled)

12. The isolated or purified Ab, or Ag binding fragment thereof, or monomeric or dimeric ABP, of claim 1, wherein the light chain variable region (VL) comprises
an amino acid sequence at least 70% identical to the amino acid sequence of SEQ ID NO:2.

13. (canceled)

14. (canceled)

15. The isolated or purified Ab, or Ag binding fragment thereof, or monomeric or dimeric ABP, of claim 1, wherein the light chain variable region (VL) further comprises at least a portion of a light chain constant region.

16. The isolated or purified Ab, or Ag binding fragment thereof, or monomeric or dimeric ABP, of claim 15, wherein the light chain constant region comprises an amino acid sequence at least 70% identical to the amino acid sequence of SEQ ID NO: 8.

17-21. (canceled)

22. The isolated or purified Ab, or Ag binding fragment thereof, or monomeric or dimeric ABP, of claim 6, wherein the heavy chain constant region comprises amino acid sequence from a IgG, IgM, IgA, IgD or IgE isotype.

23. The isolated or purified Ab, or Ag binding fragment thereof, or monomeric or dimeric ABP, of claim 15, wherein the light chain constant region comprises amino acid sequence from a kappa or lambda isotype.

24-26. (canceled)

27. The isolated or purified Ab, or Ag binding fragment thereof, or monomeric or dimeric ABP, of claim 1, further comprising a detectable protein, a detectable agent or a binding moiety.

28. (canceled)

29. (canceled)

30. The isolated or purified Ab, or Ag binding fragment thereof, or monomeric or dimeric ABP, of claim 27, wherein the detectable agent or binding moiety comprises a biotin, a fluorescent or chemiluminescent label, a fluorophore, perylene, fluorenyl, coumarin, 7-methoxy coumarin (Mca), 4-(dimethylaminoazo)benzene-4-carboxylic acid (dabcyl), Tamra, boron-dipyrromethene (BODIPY), or derivatives thereof, a dye, a cyanine dye, a Cy3, a Cy5, rhodamine, [2-(4-nitro-2,1,3-benzoxadiazol-7-yl)aminoethyl]trimethylammonium (NBD), nile red or nile blue, a fluorescent dye comprising sulfoindocyanine, a dansyl, a fluorescein, a carboxyfluorescein (FAM), a 6-FAM moiety, a theophylline, a digoxigenin, a carborane, a fluorescein, a bromodeoxyuridine moiety, a radioisotope, a quantum dot or photoluminescent aqueous nanocrystal, a hapten, or an antibody binding epitope or domain.

31-35. (canceled)

36. A chimeric or recombinant nucleic acid comprising a nucleic acid sequence encoding the Ab, or Ag binding fragment thereof, or monomeric or dimeric ABP of claim 1.

37-41. (canceled)

42. An expression cassette, a vector, a recombinant virus, an artificial chromosome, a cosmid or a plasmid comprising or having contained therein a chimeric or the recombinant nucleic acid of claim 36.

43. A cell comprising or having contained therein: the chimeric or recombinant antibody or dimeric antigen binding protein of claim 1.

44. (canceled)

45. (canceled)

46. A method for detecting the presence of a human ICOS (Inducible T-cell costimulatory) protein or polypeptide, in or on a cell, a tissue, an organ or a portion of any of the foregoing, comprising:
(a) contacting the cell, tissue or organ or portion of any of the foregoing with at least one Ab, or Ag binding fragment thereof, or monomeric or dimeric ABP of any of claim 1, and
(b) detecting the specific binding of the at least one Ab, or Ag binding fragment thereof, or monomeric or dimeric ABP with a BCMA polypeptide, in or on the cell, tissue or organ or portion of any of the foregoing, thereby detecting the presence of the human BEMA-ICOS protein in or on the cell, tissue, organ or portion of any of the foregoing.

47-61. (canceled)

62. A method for detecting or diagnosing a cancer or a tumor, wherein the method comprises detecting expression or presence of a human a human ICOS (Inducible T-cell costimulatory) protein or polypeptide, in or on a cell, tissue or organ sample using the method of claim 46, wherein the detecting of the specific binding of the Ab, or Ag binding fragment thereof, or monomeric or dimeric ABP with the human ICOS (Inducible T-cell costimulatory) protein, or the human CD278 protein or polypeptide in or on the cell, tissue or organ or portion of any of the foregoing, detects or diagnoses, or assists in the detection or diagnosis of, the cancer or the tumor.

63-72. (canceled)

73. A method for treating, ameliorating or preventing a cancer or tumor comprising first detecting or diagnosing the cancer or tumor using a method of claim 62, followed by treatment of the individual in need thereof for the treatment, amelioration or prevention of the cancer or tumor.

74-93. (canceled)

94. A kit comprising a chimeric or recombinant antibody of claim 1.

95. (canceled)
